(19) United States
(12) Patent Application Publication
(10) Pub. No.: US 2010/0288665 A1
(43) Pub. Date: Nov. 18, 2010

(54) THERAPEUTIC FORMULATIONS FOR THE TREATMENT OF COLD AND FLU-LIKE SYMPTOMS

(75) Inventor: Mark Andrew Lomaga, Totonto (CA)

Correspondence Address: HUDAK, SHUNK & FARINE, CO., L.P.A., 2020 FRONT STREET, SUITE 307, CUYAHOGA FALLS, OH, 44221, US

(73) Assignee: Kingsway Pharmaceuticals Inc., Toronto (CA)

(21) Appl. No.: 12/452,838

(22) PCT Filed: Jul. 23, 2008
(86) PCT No.: PCT/CA2008/001355

§ 371 Date: Mar. 25, 2010

Related U.S. Application Data
(60) Provisional application No. 60/935,012, filed on Jul. 23, 2007.

(51) Int. Cl. A61B 19/00; A61K 31/192; A61K 31/167; A61K 31/137; A61P 29/00; A61P 27/14; A61P 27/16; B65D 85/00

(52) U.S. Cl. 206/438; 514/570; 514/629; 514/653; 206/459.5

(57) ABSTRACT

A pharmaceutical formulation of therapeutically effective amounts of acetaminophen, ibuprofen, and a sympathomimetic drug, such as pseudoephedrine (or its prodrug), or phenylephrine used in the treatment of cold and flu-like symptoms. Such symptoms may include fever, pain, nasal congestion, sinus congestion, runny nose, sore throat, myalgia, ear pressure and fullness, and headache. The formulation further includes various excipients used in the formulation process.

### FIELD OF THE INVENTION

This invention relates to pharmaceutical formulations. More particularly, this invention relates to pharmaceutical formulation comprising a phenylpropionate, an acetanilide, and a sympathomimetic drug, and a pharmaceutically acceptable carrier.

### BACKGROUND OF THE INVENTION

Fever is a common and frequent medical symptom that describes an abnormal elevation of body temperature, usually as a result of a pathologic process. Although fever itself is probably a protective physiologic response, under certain circumstances it has the potential to be harmful. Fever increases the metabolic rate approximately 10% for every 1-degree rise in body temperature. All patients, including some children, may not tolerate the increased alterations in myocardial demand, orthostatic dysfunction, and increases in oxygen consumption, respiratory minute volume, and respiratory quotient that occur. There are many known treatments for fever.

Situations associated with fever and rhinitis include (but not limited to): the common cold, influenza, sinusitis and measles.

The cold is the most commonly occurring illness in the entire world, with more than 1 billion colds per year reported in the United States alone. The common cold is a self-limiting illness caused by any 1 of more than 200 viruses.

The common cold produces mild symptoms usually lasting 5-10 days. In contrast, the “flu” (influenza), which is caused by a different class of virus, can have severe symptoms. Of the viruses that cause a cold, the most commonly occurring subtype is a group that lives in the nasal passages known as the “rhinovirus.” Other less common cold viruses include coronavirus, adenovirus, and respiratory syncytial virus (RSV).

In the setting of conditions such as the common cold and influenza (the flu), fever is often associated with nasal congestion, sinus congestion, runny nose, earache, headache and muscle aches (myalgia).

### Acetaminophen

Acetaminophen (or paracetamol) is a known analgesic and synthetic nonopiate derivative of para-aminophenol that is used to relieve fever, headaches, and other minor aches and pains. Its IUPAC structure is N-(4-hydroxyphenyl)ethanamide and it is an anti-pyretic drug that belongs to the family of acetanilides. It is a weak, reversible, isoform-non-specific cyclooxygenase inhibitor that inhibits the formation and release of prostaglandins and is used alone in the treatment of pain and fever. The drug acts on the hypothalamus to produce antipyresis. Heat dissipation is increased as a result of vasodilatation and increased peripheral blood flow. The inhibitory effect of acetaminophen on cyclooxygenase-1 is limited, and the drug does not inhibit platelet function. In children or teenagers with a suspected viral illness, use of acetaminophen (not aspirin) is recommended because use of salicylates in these patients may be associated with an increased risk of developing Reye's syndrome.

Acetaminophen consists of a benzene ring core, substituted by one hydroxyl group and the nitrogen atom of an amide group in the para (1,4) pattern. The amide group is in fact acetamide (ethanamide). It is an extensively conjugated system, as the lone pair on the hydroxyl oxygen, the benzene pi cloud, the nitrogen lone pair, the p orbital on the carbonyl carbon and the lone pair on the carbonyl oxygen are all conjugated. The presence of two activating groups also make the benzene ring highly reactive towards electrophilic aromatic substitution. As the substituents are ortho, para directing and para with respect to each other, all positions on the ring are more or less equally activated. The conjugation also greatly reduces the basicity of the oxygens and the nitrogen, while making the hydroxyl acidic through delocalisation of charge developed on the phenoxide anion. The pKa for acetaminophen is 9.38.

Acetaminophen is a major ingredient in numerous cold and flu medications, as well as many prescription analgesics. It is considered safe for human use in recommended doses, but because of its wide availability, deliberate or accidental overdoses are fairly common.

Acetaminophen is rapidly and almost completely absorbed from the gastrointestinal (GI) tract following oral administration. After oral administration of immediate release acetaminophen preparations, peak plasma concentrations are attained within 10 to 60 minutes. Food may delay slightly the absorption of extended-release acetaminophen preparations. Acetaminophen has a plasma half-life of 1.2-3.0 hours. About 80-85% of the acetaminophen in the body undergoes conjugation principally with glucuronic acid and to a lesser extent with sulphuric acid. Microsomal enzymes in the liver also metabolize it. Acetaminophen is excreted in urine principally as acetaminophen glucuronide with small amounts of acetaminophen sulfate and mercaptate and unchanged drug. The recommended dose of acetaminophen is 12 to 15 mg/kg every 6 hours.

Known preparations that contain acetaminophen include: (oral capsules, solutions, suspensions, tablets (chewable, extended-release film coated, orally disintegrating), and rectal suppositories.

### Ibuprofen

Ibuprofen is a known prototypical nonsteroidal anti-inflammatory drug (NSAID) that also exhibits antipyretic activity. It is a propionic acid derivative that inhibits both cyclooxygenase-1 (COX-1) and -2 (COX-2), hence impairing the biosynthesis of prostaglandins in the arachidonic acid pathway. Its IUPAC structure is 2-[4-(2-methylpropyl)phenyl] propanoic acid and it belongs to the family of phenylpropionate drugs. The pKa for ibuprofen is 4.91.

Ibuprofen has been used for relief of symptoms of arthritis, primary dysmenorrhoea, fever, and as an analgesic, especially where there is an inflammatory component. Ibuprofen has also been shown to inhibit the reactivation of latent herpes simplex virus (HSV). Herpes reactivation is common with febrile episodes.

Approximately 80% of an oral dose of ibuprofen is absorbed from the GI tract. Absorption rate is slower and plasma concentrations are reduced when taken with food, however the extent of absorption is not affected. When the drug is administered with food, peak plasma ibuprofen concentrations are reduced by 30-50% and time to achieve peak plasma concentrations are reduced by 30-60 minutes. Time to reach maximal plasma concentration following administration of conventional tablets is approximately 120 minutes. In children, the antipyretic effect of ibuprofen suspension begins within 1 hour after oral administration and peaks within 2-4 hours. The antipyretic effect of single ibuprofen suspension doses of 10-mg/kg may last up to 8 hours. The plasma half-life of ibuprofen has been reported to be 2-4 hours. Ibuprofen is metabolized via oxidation to form 2 inactive metabolites that are excreted in urine. Excretion of ibuprofen is essentially complete within 24 hours following oral administration. The recommended dose is 5 to 10 mg/kg every 8 hours.

Known preparations that contain ibuprofen include: oral capsules (liquid filled); suspensions, tablets, chewable tablets, and film-coated tablets.

### Pseudoephedrine

It is known that pseudoephedrine is a sympathomimetic amine that acts directly on both α- and, to a lesser degree, β-adrenergic receptors. It is believed that α-adrenergic effects result from the inhibition of the production of cyclic adenosine-3,5-monophosphate (cAMP) by inhibition of the enzyme adenyl cyclase, whereas β-adrenergic effects result from stimulation of adenyl cyclase activity. Pseudoephedrine also has an indirect effect by releasing norepinephrine from its storage sites. The pKa for pseudoephedrine hydrochloride is 9.22.

Pseudoephedrine acts directly on α-adrenergic receptors in the mucosa of the respiratory tract producing vasoconstriction that results in shrinkage of swollen nasal mucous membranes, reduction of tissue hyperemia, edema, and nasal congestion, and an increase in nasal airway patency; drainage of sinus secretions is increased. Sympathomimetic effects of pseudoephedrine presumably also may occur in other areas of the respiratory tract, including the Eustachian tube; these effects may improve or maintain Eustachian tube patency and allow equilibration of middle ear pressure during external atmospheric pressure changes (e.g., during descent of an aircraft, underwater diving, hyperbaric oxygenation).

Numerous uses of pseudoephedrine are known and include: nasal decongestant for self-medication for the temporary relief of nasal congestion associated with upper respiratory allergy (e.g. hay fever) or the common cold; temporary relief of sinus congestion and pressure; and, symptomatic prevention or treatment of otic barotrauma.

Pseudoephedrine is almost completely absorbed from the GI tract. Following oral administration of a 60- or 120-mg dose of pseudoephedrine as an oral solution, peak plasma concentrations are achieved in approximately 1.39-2.0 or 1.84-1.97 hours, respectively. Absorption from extended-release products is slower and peak plasma concentrations of the drug are achieved in about 3.8-6.1 hours. Food delays absorption of the drug but appears not to have an effect on absorption when the drug is administered as extended-release preparations.

After oral administration of 30 mg of pseudoephedrine hydrochloride as tablets or oral solution, nasal decongestion occurs within 30 minutes and persists for 4-6 hours. Nasal decongestion may persist for 8 hours following oral administration of 60 mg and up to 12 hours following 120 mg of the drug in extended-release capsules.

Pseudoephedrine is incompletely metabolized (less than 1%) in the liver by N-demethylation to an inactive metabolite. The drug and its metabolite are excreted in urine; 55-96% of a dose is excreted unchanged. The elimination half-life of pseudoephedrine ranges from 3-6 or 9-16 hours when urinary pH is 5 or 8, respectively, while when urinary pH is 5.8, the elimination half-life of the drug ranges from 5-8 hours. With alkalinization of urine, some of the drug is reabsorbed in the kidney tubule and the rate of urinary excretion is slowed.

There has been an increase in the illegal production of methamphetamine through clandestine methods using the ephedrine or pseudoephedrine reduction method. Users could purchase over-the-counter cold and allergy tablets containing ephedrine or pseudoephedrine, place them in a solution of water, alcohol, or other solvent for several hours until the ephedrine or pseudoephedrine is separated out of the tablet. Using common household products and recipes readily available on the Internet, the ephedrine or pseudoephedrine is converted into methamphetamine. In response to this activity, the US government has enacted the Combat Methamphetamine Epidemic Act of 2005. This act bans over-the-counter sales of cold medicines that include pseudoephedrine to behind the counter as of Sep. 30, 2006.

Known preparations that contain pseudoephedrine include oral solutions, tablets, extended-release tablets, chewable tablets, extended-release core with immediate release tablets, and extended-release film-coated tablets.

### Phenylephrine

In response to the issue of misuse of pseudoephedrine-containing products, many companies are voluntarily reformulating their products to exclude pseudoephedrine and are using phenylephrine as a substitute product. However, studies have shown that the phenylephrine is less efficacious than pseudoephedrine (Hatton R C et al, The Annals of Pharmacotherapy, 2007 March, Vo. 41).

Phenylephrine acts predominantly by a direct effect on an α-adrenergic receptors (when therapeutic doses used). It is believed that α-adreneregic effects result from the inhibition of the production of cyclic adenosine-3,5-monophosphate (cAMP) by inhibition of the enzyme adenyl cyclase, whereas β-adrenergic effects result from stimulation of adenyl cyclase activity. Phenylephrine also has an indirect effect by releasing norephinephrine from its storage sites. The main effect of therapeutic doses of phenylephrine is vasoconstriction. The pKa for phenylephrine hydrochloride is 8.86.

Phenylephrine is completely absorbed following oral administration and undergoes extensive first-pass metabolism in the intestinal wall and liver. The bioavailability of phenylephrine following oral administration is approximately 38%. Following oral administration, peak serum concentrations occur at 0.75-2.0 hours and nasal decongestion may occur within 15-20 minutes and may persist for 2-4 hours.

Phenylephrine and its metabolites are excreted mainly in urine. The elimination half-life of phenylephrine is 2-3 hours.

Known preparations that contain phenylephrine include: ophthalmic solutions, oral solutions and tablets.

### Combination Products

Known formulations of acetaminophen include: acetaminophen, aspirin and caffeine; acetaminophen, ephedrine sulphate; acetaminophen, pseudoephedrine hydrochloride and chlorpheniramine. (Se Pu. 2001 May; 19(3):236-8. Chinese.); acetaminophen, dextromethorphan and doxylamine succinate (J Int Med Res. 1978; 6(2):161-5); acetaminophen, pseudoephedrine hydrochloride and triprolidine hydrochloride. (J Pharm Biomed Anal. 1994 March; 12(3):379-82); acetaminophen, ephedrine hydrochloride and caffeine (Hua Xi Yi Ke Da Xue Xue Bao. 1995 December; 26(4):443-6.); acetaminophen and codeine phosphate; acetaminophen and diphenhydramine citrate; acetaminophen, ephedrine sulfate, dextromethorphan hydrobromide and doxylamine succinate. (Int J Clin Pharmacol Ther. 2007 April; 45(4):230-6.); acetaminophen and oxycodone; acetaminophen, clemastine fumarate, and pseudoephedrine hydrochloride (Ann Allergy Asthma Immunol. 2003 January; 90(1):79-86); acetaminophen, dexbrompheniramine maleate, and pseudoephedrine sulfate; acetaminophen and pseudoephedrine hydrochloride (Arch Fam Med. 2000 November-December; 9(10):979-85); and acetaminophen and propoxyphene hydrochloride.

Known formulations of ibuprofen include: ibuprofen with pseudoephedrine hydrochloride (U.S. Pat. No. 4,552,899); ibuprofen, chlorpheniramine maleate, and pseudoephedrine; ibuprofen and diphendydramine citrate; and ibuprofen with hydrocodone bitartrate.

Acetaminophen and ibuprofen may be safely used together because the two medications have significantly different pathways of metabolism that are not affected by each other, and have been used abroad in combination form for over a decade. Both acetaminophen and ibuprofen have been shown to be safe when given individually or together in recommended doses for short-term use. There are no reports of adverse effects from combination therapy with standard doses. It was recently reported that more than 50% of pediatricians advise parents to alternate acetaminophen and ibuprofen in an attempt to achieve maximal antipyresis. In addition, other studies have reported that more than 50% of parents or caregivers give their children both antipyretics, but their method of alternation varies. Furthermore, in about half of the cases, the dosage used is inaccurate and combined overdosage with both drugs has occurred.

Formulations of acetaminophen and ibuprofen are known for the treatment of pain (Cdn. Patent Application No. 2,570,474), the treatment of fever (U.S. Pat. No. 5,409,709), and the treatment of fever in children (Clinical Trial No. NCT00267293). U.S. Pat. No. 5,409,709 teaches the combination of ibuprofen and acetaminophen used in the treatment of fever, provides no suggestions on how the combination could be used in a formulation either alone or with other pharmaceutical agents to treat the battery of conditions also found in patients suffering from cold and flu symptoms, such as nasal congestion, sinus congestion, runny nose, earache, headache and muscle aches.

U.S. Pat. No. 4,552,889 discloses a formulation of NSAIDS such as ibuprofen and pseudoephedrine used in the treatment of cold and flu-like symptoms, sold under the name ADVIL COLD AND SINUS™. Similarly, the product sold as SINUTAB™ further discloses a formulation of acetaminophen and pseudoephedrine also used in the treatment of cold and flu-like symptoms. Neither of the two formulations overcome the limitations of slow onset of action or reduced duration of action, respectively, for the treatment of cold and flu-like symptoms.

Acetaminophen and ibuprofen exert their effects at differing points in pyrogenic pathways so synergistic action is plausible. In fact, a randomised controlled trial (Erlewyn-Lajeunesse et al.) demonstrated that combined acetaminophen and ibuprofen were better at reducing fever after one hour than acetaminophen alone. Another randomised, double blind study (Sarrell et al.) of acetaminophen, ibuprofen, or both alternating demonstrated that the alternating use of acetaminophen and ibuprofen every 4 hours reduces fever faster and for a longer duration than either agent alone with no increase in adverse events.

In particular, formulations of acetaminophen and pseudoephedrine; acetaminophen and phenylephrine; ibuprofen and pseudoephedrine; and ibuprofen and phenylephrine have been shown to be safe and effective in temporarily relieving nasal congestion, sinus congestion, and pressure.

Due to the recent concerns with the abuse, misuse, and toxicity of pseudoephedrine-derived methamphetamine products, additional formulations including pseudoephedrine are less likely to be pursued. In addition, the well-documented reduced efficacy of phenylephrine as compared to pseudoephedrine would also not be a likely drug to use in creating a formulation product.

Drug absorption is one of the primary considerations when developing drug formulations, as in order for a drug to exert its biologic effect, it must be available to the target areas for interaction, resulting in alteration of cellular function. Drugs that have different ionization or dissociation constants are not likely to readily appear in formulations. For example, the different pKas of acetaminophen (9.38), ibuprofen (4.91), and pseudoephedrine hydrochloride (9.22) would not readily lead to the formulation of the three in one drug. Similarly, the different pKas of acetaminophen (9.38), ibuprofen (4.91), and phenylephrine hydrochloride (8.86) would also not readily lead to the apparent formulation of the three products in one drug.

### Prodrugs

A prodrug is an active drug chemically transformed into a per se inactive derivative which by virtue of chemical or enzymatic attack is converted to the parent (active) drug within the body before or after reaching the site of action. The process of converting an active drug into inactive form is called drug latentiation. Prodrugs can be carrier-linked-prodrugs and bioprecursors. The carrier-linked prodrug results from a temporary linkage of the active molecule with a transport moiety. Such prodrugs are less active or inactive compared to the parent active drug. The transport moiety will be chosen for its non-toxicity and its ability to ensure the release of the active principle with efficient kinetics. Whereas the bioprecursors result from a molecular modification of the active principle itself by generation of a new molecule that is capable of being a substrate to the metabolizing enzymes releasing the active principle as a metabolite.

Prodrugs are prepared to alter the drug pharmacokinetics, improve stability and solubility, decrease toxicity, increase specificity, and increase duration of the pharmacological effect of the drug. By altering pharmacokinetics the drug bioavailability is increased by increasing absorption, distribution, biotransformation, and excretion of the drug. Limited intestinal absorption, distribution, fast metabolism, and toxicity are some of the causes of failure of drug candidates during development. Avoidance of the foreseeable or proven pharmacokinetic defects thus assumes considerable significance in drug research. Accordingly, prodrugs play a significant role in drug research as well.

In designing the prodrugs, it is important to consider the following factors: a) the linkage between the carrier and the drug is usually a covalent bond, b) the prodrug is inactive or less active than the active principle, c) the prodrug synthesis should not be expensive, d) the prodrug has to be reversible or bioreversible derivative of the drug, and e) the carrier moiety must be non-toxic and inactive when released.

Prodrugs are usually prepared by: a) formation of ester, hemiesters, carbonate esters, nitrate esters, amides, hydroxamic acids, carbamates, imines, mannich bases, and enamines of the active drug, b) functionalizing the drug with azo, glycoside, peptide, and ether functional groups, c) use of polymers, salts, complexes, phosphoramides, acetals, hemiacetals, and ketal forms of the drug. For example, see Andrejus Korolkovas's, “Essentials of Medicinal Chemistry”, pp. 97-118.

Canadian Patent No. 2,540,678 teaches a method of preventing overdose by protecting single drug products with a prodrug, such as lysine. This patent does not disclose how prodrugs such as lysine can be used with combination formulations to exert similar effects.

Thus, there remains a need in the art to treat the various symptoms of cold and flu in one drug which allow for the effective relief of cold symptoms treated by sympathomimetic drugs such as pseudoephedrine and phenylephrine, also while exerting effects at differing points in the pyrogenic pathways in the treatment of fever, headaches, and aches and pains.

It is further desired to have an improved method of delivering the drug formulation in such a way that limits the ability of abusers of drugs such as pseudoephedrine through alternate routes of administration or extraction techniques. Also, it is desired to have pseudoephedrine containing products available over-the-counter to improve access to consumers, and reduce workload on pharmacists who document amounts purchased.

It is desired to have a formulation of drugs, which use different features along the pyrogenic pathways, to more effectively result in a faster onset and longer lasting treatment of fever and various other cold and flu symptoms.

It is further desired to have a formulation of drugs used for the treatment of cold and flu-like symptoms, such as: sore throat, and/or muscle pain, and/or fever, and/or nasal congestion, and/or sinus congestion, and/or runny nose, and/or myalgia, and/or otic barotrauma. Very often, these symptoms manifest together and it is desired to have one drug rather than multiple drugs used in the treatment of these symptoms. For example, with influenza, one often has muscle pain, fever, headache, and runny nose. Using phenylpropionates and acetonilides in combination it is believed to treat fever, muscle pain, and headaches (via pharmacokinetic and pharmacodynamic advantages) more effectively than when used individually. Using sympathomimetics alone will effectively treat runny nose, nasal congestion, sinus congestion, and otic fullness, but not fever, headache, and pain.

### SUMMARY OF THE INVENTION

The present invention comprises a pharmaceutical formulation comprising a phenylpropionate, an acetanilide, and a sympathomimetic drug, and a pharmaceutically acceptable carrier.

The present invention further comprises a pharmaceutical formulation comprising, ibuprofen, acetaminophen, pseudoephedrine, and a pharmaceutically acceptable carrier.

The present invention also comprises a pharmaceutical formulation comprising, ibuprofen, acetaminophen, and a prodrug of pseudoephedrine (such as lysine), and a pharmaceutically acceptable carrier.

The present invention also comprises a pharmaceutical formulation comprising, ibuprofen, acetaminophen, phenylephrine, and a pharmaceutically acceptable carrier.

The present invention also comprises the use of the pharmaceutical formulations stated above for the treatment of at least one of pain, headache, fever, nasal congestion, sinus congestion, runny nose, sore throat, myalgia, or otic barotrauma.

The present invention also comprises a dosing regimen for any of the pharmaceutical formulations listed above.

The present invention further comprises a method of treating one or more of the following symptoms comprising pain, headache, fever, nasal congestion, sinus congestion, runny nose, or otic barotrauma, where the method involves administering to a mammalian subject any of the formulations listed above.

The present invention further comprises a method of treating one or more of the following symptoms comprising pain, headache, fever, nasal congestion, sinus congestion, runny nose, sore throat, myalgia, or ear fullness, where the method involves administering to a mammalian subject suffering from those conditions the dosing regimen listed above.

The present invention further comprises a kit comprising three or more pharmaceutical formulations, wherein the first pharmaceutical formulation comprises a phenylpropionate and a pharmaceutically acceptable carrier, the second pharmaceutical formulation comprises an acetanilide and a pharmaceutically acceptable carrier, and the third pharmaceutical formulation comprises a sympathomimetic drug and a pharmaceutically acceptable carrier.

The present invention further comprises a pharmaceutical formulation comprising a phenylpropionate, such as ibuprofen, a modified sympathomimetic prodrug, such as pseudoephedrine, linked with a peptide, such as lysine.

The present invention further comprises a pharmaceutical formulation comprising an acetanilide, such as acetaminophen, a sympathomimetic prodrug, such as pseudoephedrine, linked with a peptide, such as lysine.

It is an object of the present invention to provide for a novel treatment of any one or a combination of the following conditions: pain, headache, fever, nasal congestion, sinus congestion, runny nose, sore throat, myalgia, or otic barotrauma. The present invention offers a solution to the problems associated with the current products on the market, namely a formulation of drugs comprising three active components that more effectively result in a faster onset and longer lasting treatment of fever and various other cold and flu symptoms.

It is a further object of the invention to provide a prodrug, such as a peptide or a combination of multiple prodrugs (or multiple peptides), which reduce the ability to extract pseudoephedrine in the illicit business of methamphetamine production.

### DETAILED DESCRIPTION OF THE INVENTION

It has been discovered that the combination of phenylpropionates, acetonilides, and sympathomimetic drugs (including relevant prodrugs), enhance the efficacy of treating cold and flu-like symptoms through faster onset and longer duration of action of pharmacologic effect. The pharmaceutical compositions of this invention comprise a group of drugs whose main action is pronounced and results in the treatment of pain, and/or fever (pyrexia), and/or nasal congestion, and/or sinus congestion, and/or runny nose, and/or otic barotrauma, and/or myalgia, and/or headache. Namely, this combination includes therapeutically effective amounts of acetaminophen, ibuprofen, pseudoephedrine or a pharmaceutically acceptable salt thereof, and a carrier. This discovery is particularly advantageous because combining the three classes of drugs is likely to circumvent problems associated with inaccurate and combined under- and overdosages by the manufacturing of standard dosages. This would improve compliance, safety, and efficacy, as well as reducing costs to the patient and the health care system.

Cold and flu-like symptoms as used herein refer to coryzea, nasal congestion, upper respiratory infections, allergic rhinitis, ear fullness, sinusitis, and the like. Runny nose and nasal congestion can also be cold symptoms.

The terms “effective amount” or “therapeutically effective amount” of an active agent as provided herein is defined as an amount of the agent at least sufficient to provide the desired therapeutic effect. The exact amount required will vary from subject to subject, depending on age, general condition of the subject, the severity of the condition being treated, and the particular active agent administered, and the like.

The term “normal approved dose” of an active agent as provided herein is defined as an amount of the agent that has been approved as safe and effective by the United States Food and Drug Administration for administration in humans in a particular dosage form. An approved dose is thus a dose found in a pharmaceutical product, an amount of active agent per unit dosage form. In the present invention, reference to a ratio of approved doses means doses approved for the same patient population (e.g., adult to adult or pediatric to pediatric), and approved for the same dosage form (e.g., elixir, tablet, capsule, caplet, controlled release, etc.).

The first component of the drug combination of this invention is a phenylpropionate. Useful phenylpropionates include non-steroidal anti-inflammatory drugs (NSAIDS) such as ibuprofen and its salts, esters, and other complexes. Phenylpropionates such as ibuprofen are used in the treatment of fever and pain when present in therapeutically effective amounts. Phenylpropionates inhibit both cyclooxygenase-1 (COX-1) and -2 (COX-2), hence impairing the biosynthesis of prostaglandins in the aracidonic acid pathway.

The second component of the drug combination of this invention is an acetanilide. Useful acetanilides include acetaminophen and its salts, esters, and other complexes. Acetanilides such as acetaminophen are used in the treatment of fever and pain when present in therapeutically effective amounts. Unlike phenylpropionates, acetanilides such as acetaminophen are weak, reversible, isoform-non-specific cyclooxygenase inhibitors that inhibit the formation and release of prostaglandins and are used alone in the treatment of pain and fever. The drug acts on the hypothalamus to produce antipyresis. Heat dissipation is increased as a result of vasodilatation and increased peripheral blood flow. The inhibitory effect of acetanilides such as acetaminophen on cyclooxygenase-1 is limited, and the drug does not inhibit platelet function. Thus, both acetanilides and phenylpropionates have similar therapeutic indications and pharmacodynamic effects, but have different pharmacokinetics (absorption, distribution, metabolism, and elimination). Because they exert their differences at different points in pyrogenic pathways, synergistic action is plausible.

The third component of this drug composition is a sympathomimetic drug used as a decongestant. The decongestants for use in the pharmaceutical compositions and methods of use of the present invention include, but are not limited to, pseudoephedrine, pseudoephedrine salts, including pseudoephedrine hydrochloride and pseudoephedrine sulfate, phenylephrine, phenylephrine salts, including phenylephrine hydrochloride, and phenylpropanolamine. One of skill in the art would know of many other appropriate decongestants and their approved dosages.

Pseudoephedrine drugs acts directly on both α- and to a lesser degree, β-adrenergic receptors. It is believed that α-adrenergic effects result from the inhibition of the production of cyclic adenosine-3,5-monophosphate (cAMP) by inhibition of the enzyme adenyl cyclase, whereas β-adrenergic effects result from stimulation of adenyl cyclase activity. Phenylephrine also has an indirect effect by releasing norepinephrine from its storage sites.

Pseudoephedrine acts directly on α-adrenergic receptors in the mucosa of the respiratory tract producing vasoconstriction that results in shrinkage of swollen nasal mucous membranes, reduction of tissue hyperemia, edema, and nasal congestion, and an increase in nasal airway patency; drainage of sinus secretions is also increased.

Sympathomimetic effects of pseudoephedrine presumably may also occur in other areas of the respiratory tract, including the Eustachian tube. These effects may improve or maintain Eustachian tube patency and allow equilibration of middle ear pressure during external atmospheric pressure changes (e.g. during descent of an aircraft, underwater diving, hyperbaric oxygenation).

If pseudoephedrine is linked to a peptide, such as lysine or a combination of peptides, can also act as a prodrug. In the prodrug form, extraction is prevented since bioactivation by gut enzymes are in vivo and are required to cleave lysine from pseudoephedrine, thereby rendering methamphetamine extraction possible only in vivo and not in vitro.

Phenylephrine acts predominantly by a direct effect on α-adrenergic receptors, when therapeutic doses are used. It is believed that α-adrenergic effects result from the inhibition of the production of cyclic adenosine-3,5-monophosphate (cAMP) by inhibition of the enzyme adenyl cyclase, whereas β-adrenergic effects result from stimulation of adenyl cyclase activity. Phenylephrine also has an indirect effect by releasing norepinephrine from its storage sites. The main effect of therapeutic doses of phenylephrine is vasoconstriction.

Phenylephrine is completely absorbed following oral administration and undergoes extensive first-pass metabolism in the intestinal wall and liver. The bioavailabilty of phenylephrine following oral administration is approximately 38%. Following oral administration, peak serum concentrations occur at 0.75-2.0 hours and nasal decongestion may occur within 15-20 minutes and may persist for 2-4 hours.

The amounts of first, second, and third components present in a unit dose of the drug composition can be the same as those employed in comparable dosage forms of known drugs such as those previously mentioned. Suitable amounts can be readily determined by using routine procedures. In general, therapeutically effective amounts of phenylpropionates may range from 5-10 mg/kg every 8 hours. Therapeutically effective amounts of acetonitriles may range from 10-15 mg/kg every 6 hours. Therapeutically effective amounts of phenylephrine hydrochloride may range from 5 to 10 mg every 4 hours in adults and children 12 years of age an older. Maximum recommended daily dose is 60 mg. Therapeutically effective amounts of pseudoephedrine hydrochloride for adults and children over 12 years of age is 60 mg every 4-6 hours with a maximum dosage of 240 mg daily. Children 6 to 11 years of age may receive 30 mg every 4-6 hours, with a maximum dosage of 120 mg daily, and children 2-5 years of age may receive 15 mg every 4-6 hours with a maximum dosage of 60 mg daily. With extended release, adults and children 12 years of age or older may receive 120 mg every 12 hours.

The present invention circumvents problems associated with inaccurate and combined under- and overdosages by the manufacturing of standard dosages. This in turn would improve compliance, safety and efficacy, and also would reduce costs transferred to patients and/or the health care system.

Other advantages are pharmacokinetic-based, as acetaminophen results in fast onset of therapeutic amount and ibuprofen resulting in a longer duration but with a slower onset. Also, food does not affect absorption with acetaminophen to the same extent as with ibuprofen. Fewer dosages would need to be taken, making it a more cost effective option with greater ease of use. By decreasing the interval of doses, patient compliance is increased, thus resulting in a reduction in cost.

Since fever is often associated with nasal congestion, sinus congestion and/or runny nose, the added use of a decongestant such as pseudoephedrine offers superior cold and flu-like symptom relief compared with a product containing only acetaminophen and ibuprofen.

Compositions of the invention are formulated in a single dosage form, and these may be solid (such as tablets, capsules, sachets, trochets and the like), liquid (such as solutions or suspensions) or inhalation aerosols or patches. While the solid compounds will typically be administered orally, the liquids may be administered orally or by injection. Other dosage forms, such as suppositories, are also useful.

The drug composition will ordinarily be formulated with one or more pharmaceutically acceptable ingredients in accordance with known and established practice. The composition can thus be formulated as a liquid, powder, elixir, suspension, gel capsules, capsules, solutions, tablets, chewable tablets, extended-release film-coated tablets, orally disintegrating tablets, liquid filled capsules. The present composition can be formulated for oral administration as a liquid or solid dosage form with immediate, slow delayed or sustained-release characteristics.

Aqueous suspensions can include pharmaceutically acceptable excipients, such as suspending agents (sodium carboxymethyl cellulose, methylcellulose, hydroxypropylmethylcellulose, sodium alginate, polyvinylpyrrolidone, gum tragacanth, and gum acacia), dispersing or wetting agents (naturally occurring phosphatide); preservatives; colouring agents, flavouring agents, sweetening agents (sucrose, saccharin, sodium cyclamate, or calcium cyclamate).

Binders are agents used to impart cohesive qualities to the powdered material. Binders impart cohesiveness to the tablet formulation which ensures tablets remain intact after compression, as well as improving the free-flowing qualities by the formulation of granules of desired hardness and size. Suitable binder materials include, but are not limited to, starch (including corn starch and pregelatinzed starch), gelatin, sugars (including sucrose, glucose, dextrose, lactose and sorbitol), polyethylene glycol, waxes, natural and synthetic gums, e.g., acacia, tragacanth, sodium alginate, celluloses, and Veegum, and synthetic polymers such as polymethacrylates and polyvinylpyrrolidone.

Lubricants have a number of functions in tablet manufacture. They prevent adhesion of the tablet material to the surface of the dyes and punches, reduce interparticle friction, facilitate the ejection of the tablets from the dye cavity and may improve the rate of flow of the tablet granulation. Examples of suitable lubricants include, but are not limited to, magnesium stearate, calcium stearate, stearic acid, glyceryl behenate, talc, sodium lauryl sulfate, sodium stearyl fumarate, polyethylene glycol or mixtures thereof. Generally, the lubricant is present in an amount from about 0.25% to about 5% of the weight of the final composition and more specifically from about 0.5 to about 1.5% of the weight of the final composition.

A disintegrant is a substance, or a mixture of substances, added to a tablet to facilitate its breakup or disintegration after administration. Materials serving as disintegrants have been classified chemically as starches, clay, celluloses, aligns, gums and cross-linked polymers. Examples of suitable disintegrants include, but are not limited to, croscarmelose sodium, sodium starch glycolate, starch, magnesium aluminum silicate, colloidal silicon dioxide, methylcellulose, agar, bentonite, alginic acid, guar gum, citrus pulp, carboxymethyl cellulose, microcrystalline cellulose, or mixtures thereof. Generally, the disintegrant is present in an amount from about 0.5% to about 25% of the weight of the final composition and more specifically from about 1% to about 15% of the weight of the final composition.

Glidants are substances which improve the flow characteristics of a powder mixture. Examples of glidants include, but are not limited to colloidal silicon dioxide, talc or mixtures thereof. Generally, the glident is present in an amount of from about 0.1% to about 10% of the weight of the final composition and more specifically from 5 about 0.1% to about 5% of the weight of the final composition.

The adsorbent may be, for example colloidal silicon dioxide, microcrystalline cellulose, calcium silicate or mixtures thereof. Generally, the adsorbent is present in an amount from about 0.05% to about 42% of the weight of the final composition and more specifically from about 0.05% to about 37% of the weight of the final composition.

If desired, other ingredients, such as diluents, stabilizers and anti-adherents, conventionally used for pharmaceutical formulations may be included in the present formulations. Optional ingredients include coloring and flavoring agents which are well known in the art.

The pharmaceutical composition described in the present invention may be formulated to release the active ingredients in a sustained release manner. Various formulations, including elixers, suspensions, tablets, caplets, capsules, and the like are contemplated for dosage forms of these components.

The invention is further described by means of the following examples, which are not intended to limit the scope of the claimed invention in any manner.

Pharmaceutical composition dosage forms of the present invention are made of the active ingredients listed below in the following dosage amounts:

| | | First | Second | Third |
|---|---|---|---|---|
| Ex. | Dosage | Component | Component | Component |
| 1 | Chewable | Ibuprofen | Acetaminophen | Pseudoephedrine |
| | tablets | 50 mg | 80 mg | 15 mg |
| 2 | Chewable | Ibuprofen | Acetaminophen | Pseudoephedrine |
| | tablets | 100 mg | 120 mg | 15 mg |
| 3 | Chewable | Ibuprofen | Acetaminophen | Pseudoephedrine |
| | tablets | 100 mg | 80 mg | 15 mg |
| 4 | Chewable | Ibuprofen | Acetaminophen | Pseudoephedrine |
| | tablets | 100 mg | 160 mg | 15 mg |
| 5 | Tablets | Ibuprofen | Acetaminophen | Pseudoephedrine |
| | | 200 mg | 325 mg | 30 mg |
| 6 | Tablets | Ibuprofen | Acetaminophen | Pseudoephedrine |
| | | 200 mg | 500 mg | 30 mg |
| 7 | Capsules | Ibuprofen | Acetaminophen | Pseudoephedrine |
| | | 200 mg | 325 mg | 30 mg |
| 8 | Capsules | Ibuprofen | Acetaminophen | Pseudoephedrine |
| | | 200 mg | 500 mg | 30 mg |
| 9 | Tablets | Ibuprofen | Acetaminophen | Pseudoephedrine |
| | | 400 mg | 325 mg | 30 mg |
| 10 | Tablets | Ibuprofen | Acetaminophen | Pseudoephedrine |
| | | 400 mg | 500 mg | 30 mg |
| 11 | Capsules | Ibuprofen | Acetaminophen | Pseudoephedrine |
| | | 400 mg | 325 mg | 30 mg |
| 12 | Capsules | Ibuprofen | Acetaminophen | Pseudoephedrine |
| | | 400 mg | 500 mg | 30 mg |
| 13 | Tablets | Ibuprofen | Acetaminophen | Pseudoephedrine |
| | | 600 mg | 325 mg | 30 mg |
| 14 | Capsules | Ibuprofen | Acetaminophen | Pseudoephedrine |
| | | 600 mg | 325 mg | 30 mg |
| 15 | Tablets | Ibuprofen | Acetaminophen | Pseudoephedrine |
| | | 200 mg | 325 mg | 60 mg |
| 16 | Tablets | Ibuprofen | Acetaminophen | Pseudoephedrine |
| | | 200 mg | 500 mg | 60 mg |
| 17 | Tablets | Ibuprofen | Acetaminophen | Pseudoephedrine |
| | | 400 mg | 325 mg | 60 mg |
| 18 | Tablets | Ibuprofen | Acetaminophen | Pseudoephedrine |
| | | 400 mg | 500 mg | 60 mg |
| 19 | Capsules | Ibuprofen | Acetaminophen | Pseudoephedrine |
| | | 200 mg | 325 mg | 60 mg |
| 20 | Capsules | Ibuprofen | Acetaminophen | Pseudoephedrine |
| | | 200 mg | 500 mg | 60 mg |
| 21 | Capsules | Ibuprofen | Acetaminophen | Pseudoephedrine |
| | | 400 mg | 325 mg | 60 mg |
| 22 | Capsules | Ibuprofen | Acetaminophen | Pseudoephedrine |
| | | 400 mg | 500 mg | 60 mg |
| 23 | Tablets | Ibuprofen | Acetaminophen | Pseudoephedrine |
| | | 200 mg | 325 mg | 120 mg |
| 24 | Tablets | Ibuprofen | Acetaminophen | Pseudoephedrine |
| | | 200 mg | 500 mg | 120 mg |
| 25 | Tablets | Ibuprofen | Acetaminophen | Pseudoephedrine |
| | | 400 mg | 325 mg | 120 mg |
| 26 | Tablets | Ibuprofen | Acetaminophen | Pseudoephedrine |
| | | 400 mg | 500 mg | 120 mg |
| 27 | Capsules | Ibuprofen | Acetaminophen | Pseudoephedrine |
| | | 200 mg | 325 mg | 120 mg |
| 28 | Capsules | Ibuprofen | Acetaminophen | Pseudoephedrine |
| | | 200 mg | 500 mg | 120 mg |
| 29 | Capsules | Ibuprofen | Acetaminophen | Pseudoephedrine |
| | | 400 mg | 325 mg | 120 mg |
| 30 | Capsules | Ibuprofen | Acetaminophen | Pseudoephedrine |
| | | 400 mg | 500 mg | 120 mg |
| 31 | Liquid | Ibuprofen | Acetaminophen | Pseudoephedrine |
| | | 100 mg/5 ml | 160 mg/5 ml | 7.5 mg/0.8 ml |
| 32 | Liquid | Ibuprofen | Acetaminophen | Pseudoephedrine |
| | | 40 mg/ml | 100 mg/ml | 7.5 mg/0.8 ml |
| 33 | Chewable | Ibuprofen | Acetaminophen | Phenylephrine |
| | tablets | 50 mg | 80 mg | 5 mg |
| 34 | Tablets | Ibuprofen | Acetaminophen | Phenylephrine |
| | | 400 mg | 325 mg | 10 mg |
| 35 | Tablets | Ibuprofen | Acetaminophen | Phenylephrine |
| | | 400 mg | 500 mg | 10 mg |
| 36 | Capsules | Ibuprofen | Acetaminophen | Phenylephrine |
| | | 400 mg | 325 mg | 10 mg |
| 37 | Capsules | Ibuprofen | Acetaminophen | Phenylephrine |
| | | 400 mg | 500 mg | 10 mg |
| 38 | Liquid | Ibuprofen | Acetaminophen | Phenylephrine |
| | | 100 mg/5 ml | 160 mg/5 ml | 10 mg |
| 39 | Liquid | Ibuprofen | Acetaminophen | Phenylephrine |
| | | 40 mg/ml | 100 mg/ml | 10 mg |
| 40 | Chewable | Ibuprofen | Acetaminophen | Pseudoephedrine |
| | tablets | 50 mg | 80 mg | 15 mg |
| | | | | Lysine |
| 41 | Chewable | Ibuprofen | Acetaminophen | Pseudoephedrine |
| | tablets | 100 mg | 120 mg | 15 mg |
| | | | | Lysine |
| 42 | Chewable | Ibuprofen | Acetaminophen | Pseudoephedrine |
| | tablets | 100 mg | 80 mg | 15 mg |
| | | | | Lysine |
| 43 | Chewable | Ibuprofen | Acetaminophen | Pseudoephedrine |
| | tablets | 100 mg | 160 mg | 15 mg |
| | | | | Lysine |
| 44 | Tablets | Ibuprofen | Acetaminophen | Pseudoephedrine |
| | | 200 mg | 325 mg | 30 mg |
| | | | | Lysine |
| 45 | Tablets | Ibuprofen | Acetaminophen | Pseudoephedrine |
| | | 200 mg | 500 mg | 30 mg |
| | | | | Lysine |
| 46 | Capsules | Ibuprofen | Acetaminophen | Pseudoephedrine |
| | | 200 mg | 325 mg | 30 mg |
| | | | | Lysine |
| 47 | Capsules | Ibuprofen | Acetaminophen | Pseudoephedrine |
| | | 200 mg | 500 mg | 30 mg |
| | | | | Lysine |
| 48 | Tablets | Ibuprofen | Acetaminophen | Pseudoephedrine |
| | | 400 mg | 325 mg | 30 mg |
| | | | | Lysine |
| 49 | Tablets | Ibuprofen | Acetaminophen | Pseudoephedrine |
| | | 400 mg | 500 mg | 30 mg |
| | | | | Lysine |
| 50 | Capsules | Ibuprofen | Acetaminophen | Pseudoephedrine |
| | | 400 mg | 325 mg | 30 mg |
| | | | | Lysine |
| 51 | Capsules | Ibuprofen | Acetaminophen | Pseudoephedrine |
| | | 400 mg | 500 mg | 30 mg |
| | | | | Lysine |
| 52 | Tablets | Ibuprofen | Acetaminophen | Pseudoephedrine |
| | | 600 mg | 325 mg | 30 mg |
| | | | | Lysine |
| 53 | Capsules | Ibuprofen | Acetaminophen | Pseudoephedrine |
| | | 600 mg | 325 mg | 30 mg |
| | | | | Lysine |
| 54 | Tablets | Ibuprofen | Acetaminophen | Pseudoephedrine |
| | | 200 mg | 325 mg | 60 mg |
| | | | | Lysine |
| 55 | Tablets | Ibuprofen | Acetaminophen | Pseudoephedrine |
| | | 200 mg | 500 mg | 60 mg |
| | | | | Lysine |
| 56 | Tablets | Ibuprofen | Acetaminophen | Pseudoephedrine |
| | | 400 mg | 325 mg | 60 mg |
| | | | | Lysine |
| 57 | Tablets | Ibuprofen | Acetaminophen | Pseudoephedrine |
| | | 400 mg | 500 mg | 60 mg |
| | | | | Lysine |
| 58 | Capsules | Ibuprofen | Acetaminophen | Pseudoephedrine |
| | | 200 mg | 325 mg | 60 mg |
| | | | | Lysine |
| 59 | Capsules | Ibuprofen | Acetaminophen | Pseudoephedrine |
| | | 200 mg | 500 mg | 60 mg |
| | | | | Lysine |
| 60 | Capsules | Ibuprofen | Acetaminophen | Pseudoephedrine |
| | | 400 mg | 325 mg | 60 mg |
| | | | | Lysine |
| 61 | Capsules | Ibuprofen | Acetaminophen | Pseudoephedrine |
| | | 400 mg | 500 mg | 60 mg |
| | | | | Lysine |
| 62 | Tablets | Ibuprofen | Acetaminophen | Pseudoephedrine |
| | | 200 mg | 325 mg | 120 mg |
| | | | | Lysine |
| 63 | Tablets | Ibuprofen | Acetaminophen | Pseudoephedrine |
| | | 200 mg | 500 mg | 120 mg |
| | | | | Lysine |
| 64 | Tablets | Ibuprofen | Acetaminophen | Pseudoephedrine |
| | | 400 mg | 325 mg | 120 mg |
| | | | | Lysine |
| 65 | Tablets | Ibuprofen | Acetaminophen | Pseudoephedrine |
| | | 400 mg | 500 mg | 120 mg |
| | | | | Lysine |
| 66 | Capsules | Ibuprofen | Acetaminophen | Pseudoephedrine |
| | | 200 mg | 325 mg | 120 mg |
| | | | | Lysine |
| 67 | Capsules | Ibuprofen | Acetaminophen | Pseudoephedrine |
| | | 200 mg | 500 mg | 120 mg |
| | | | | Lysine |
| 68 | Capsules | Ibuprofen | Acetaminophen | Pseudoephedrine |
| | | 400 mg | 325 mg | 120 mg |
| | | | | Lysine |
| 69 | Capsules | Ibuprofen | Acetaminophen | Pseudoephedrine |
| | | 400 mg | 500 mg | 120 mg |
| | | | | Lysine |
| 70 | Liquid | Ibuprofen | Acetaminophen | Pseudoephedrine |
| | | 100 mg/5 ml | 160 mg/5 ml | 7.5 mg/0.8 ml |
| | | | | Lysine |
| 71 | Liquid | Ibuprofen | Acetaminophen | Pseudoephedrine |
| | | 40 mg/ml | 100 mg/ml | 7.5 mg/0.8 ml |
| | | | | Lysine |

Additional active components to the examples provided above may include caffeine, chlorpheniramine maleate, codeine phosphate, diphenhydramine citrate, oxycodone, propoxyphene hydrochloride, butalbital, dichloralphenazone, isometheptene mucate, hydrocodone bitartrate, phenyltoloxamine citrate, tramadol hydrochloride, pamabrom, pyrilamine maleate, phenylpropanolamine hydrochloride, propoxyphene napsylate, cetrizine (REACTINE), loratidine (CLARITIN), and fexofenadine (ALLEGRA).

Further examples are as follows:

### Example 1

Formula MAL-1

| Ingredients | Mg/tablet |
|---|---|
| Acetaminophen granulation | 325 |
| Ibuprofen granulation | 200 |
| Pseudoephedrine USP | 30 |
| Pre-gelatinized corn starch N.F. | 50 |
| MCC (microcrystalline cellulose) N.F. | 40 |
| Klucel | 10 |
| Magnesium Stearate N.F. | 10 |
| TOTAL | 665 |

Film coating of the tablet should be no more than 1.5% to 2% of the core weight.

| Ingredients | Mg/tablet |
|---|---|
| Povidone (K29-32) | 0.95 |
| HPMC (hydroxypropylmethyl cellulose) | 5.05 |
| Propylene Glycol | 1.13 |
| Arlacel 20 | 0.71 |
| Tween 20 | 0.47 |
| Color White | 3.31 |
| Antifoam | 0.01 |
| TOTAL | 11.63 |

### Preparation of the Core Tablets

1. Acetaminophen, ibuprofen and pseudoephedrine are mixed in a Twin Shell Blender for 15 minutes at 15 RPM.
2. Magnesium stearate is screened through a No. 30 mesh screen and mixed with the above mixture in a Twin Shell Blender for 5 minutes.
3. Tablets are compressed on a rotary press using 12 mm×5 mm capsule shaped punches (caplets).

### Coating Procedure

A coating composition is prepared having the following formula:

| Ingredients | % (w/w) |
|---|---|
| Water | 84.00 |
| Povidone (K29-32) | 1.00 |
| HPMC | 5.34 |
| Propylene Glycol | 1.20 |
| Arlacel 20 | 0.75 |
| Tween 20 | 0.50 |
| Color White T-510W | 7.00 |
| Antifoam | 0.01 |
| TOTAL | 99.80 |

This coating is applied to the core tablets described above using the following procedure:
4. Place the 6KG-compressed caplets into 24″ Accela Cota pan and turn on the exhaust and heater. Pre-heat the caplets to 40 degrees Celsius while jogging the pan.
5. 0.132 Kg of the film coating solution is sprayed through spray gums using the following parameters:
   Pan speed: 6-8 rpm
   Spray rate: 30 ml/min
   No. of spray guns: 2
   Inlet temp: 40 degrees Celsius
   Outlet temp: 38 degrees Celsius
   Atomozation Air Pressure: 20 psi
6. At the completion of the coating, the film-coated caplets are dried while jogging the coating pan until outlet temperature reaches 42 degrees Celsius.

Under actual use conditions caplets are likely to be exposed to stress conditions, such as high humidity, which can have an effect on the dissolution rate of the actives in a caplet. With this in mind, the caplets are subjected to the ICH accelerated stability conditions as per the worldwide-recognized test.

To compare the release rates of the active ingredients of caplets embodied in the present invention (MAL-1) with comparable tablets containing acetaminophen and pseudoephedrine, and ibuprofen and pseudoephedrine, the caplets MAL-1 are stressed using the ICH accelerated guidelines.

### Dissolution Rate Test

The dissolution test calls for the use of 900 ml water maintained at 37 degrees Celsius and the USP paddle, known as Apparatus 2, rotated at 50 rpm.

The tablet is placed in the beaker of water or buffer solution (pH 7.2) and after 45 minutes of paddle rotation at 50 rpm, an aliquot of solution is analyzed for acetaminophen, ibuprofen, and pseudoephedrine content.

The analysis is done via high pressure liquid chromatography (HPLC) or via spectrophotometric analysis using a multi-component analysis on HP8450 or HP8451 spectrophotometer.

Dissolution to meet the guidelines as described in the United States Pharmacopoeia.

### Example 2

A clinical trial is conducted as follows:

### Subjects

Subjects are eligible to participate if they are otherwise healthy, have cold symptoms of 48 hours or less duration, and report at least moderate symptom severity in response to the question, “overall, how would you rate the severity of your sinus symptoms? Absent, mild, moderate, moderately severe, or severe”. The subjects are blinded to the inclusion criteria and primary outcome measures. Women of childbearing age would be required to have negative results from a pregnancy test and use effective birth control. Subjects whose diastolic blood pressure was above 90 mm Hg at the time of screening are excluded. Subjects with underlying illnesses that might be exacerbated by sympathomimetic drugs or that might affect the assessment of common cold symptoms are also excluded from the study. Subjects receiving medications that might interact with sympathomimetic drugs are also excluded from the study.

### Study Medications

Study subjects are randomly assigned to receive bottles containing either 60 mg of pseudoephedrine plus 200 mg of ibuprofen plus 500 mg of acetaminophen or identically appearing placebo tablets. A study nurse administers the initial dose of study medication at approximately 8 am and 11 am. The second dose is administered approximately 8 hours after the first dose. The third dose is self-administered approximately 8 hours after the second dose. Empty bottles are collected to confirm that all medications had been received. Subjects will be instructed to avoid any other cold and flu treatments while in the study.

### Study Procedures

The study is a randomized, double-blinded, placebo-controlled clinical trial. Prior to the first dose of study medication, baseline symptom evaluations are made using a symptom severity scale of 0 to 4, corresponding to absent, mild, moderate, moderately severe, and severe. The symptoms rated are overall nasal symptom assessment, sneezing, runny nose, nasal obstruction (stopped-up nose); overall sinus symptom assessment, sinus pain, sinus pressure, sinus congestion; overall throat symptom assessment, sore throat, cough, headache, muscle pain, and malaise. These symptom assessments are made by the subjects based on their self-perception of symptom type and location. Height and weight are recorded. Subjects are instructed to contact the study site one to two hours after taking each dose of study medication, and one to two hours before taking the next dose. The symptoms and severity scale used in these assessments are the same as those used for the baseline evaluation. If the one to two-hour and six to seven hour post-dose symptom evaluations cannot be made interactively with the study staff, the subject will record his or her assessments to the questions in writing in a diary that is provided for this purpose. Most of the assessments are performed interactively with the study staff. Subjects are told to avoid concomitant medication during the 24-hour study period. If administration of concomitant medications is unavoidable, such administration is recorded in the study diary. Subjects record the occurrence of any adverse effect. Each subject reports to the study site 24 to 48 hours after receiving the third dose of study medications. Study diaries are returned and reviewed, and study medication bottles are returned.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

For example, effective dosages other than the preferred ranges set forth herein above with respect to the active ingredients may be applicable as a consequence of variations of the responsiveness of the mammal treated, severity of symptoms, dosage related adverse effects, if any, observed and similar considerations. Accordingly, such expected variations or differences in the practice of the present invention and the results obtained are contemplated in accordance with the objects and practices of the present invention.

Similar examples can also be created and studied by using the following pharmaceutical composition dosage forms based on the active ingredients listed below:

| | | First | Second | Third |
|---|---|---|---|---|
| Ex. | Dosage | Component | Component | Component |
| 1 | Chewable | Ibuprofen | Acetaminophen | Pseudoephedrine |
| | tablets | 50 mg | 80 mg | 15 mg |
| 2 | Chewable | Ibuprofen | Acetaminophen | Pseudoephedrine |
| | tablets | 100 mg | 120 mg | 15 mg |
| 3 | Chewable | Ibuprofen | Acetaminophen | Pseudoephedrine |
| | tablets | 100 mg | 80 mg | 15 mg |
| 4 | Chewable | Ibuprofen | Acetaminophen | Pseudoephedrine |
| | tablets | 100 mg | 160 mg | 15 mg |
| 5 | Tablets | Ibuprofen | Acetaminophen | Pseudoephedrine |
| | | 200 mg | 325 mg | 30 mg |
| 6 | Tablets | Ibuprofen | Acetaminophen | Pseudoephedrine |
| | | 200 mg | 500 mg | 30 mg |
| 7 | Capsules | Ibuprofen | Acetaminophen | Pseudoephedrine |
| | | 200 mg | 325 mg | 30 mg |
| 8 | Capsules | Ibuprofen | Acetaminophen | Pseudoephedrine |
| | | 200 mg | 500 mg | 30 mg |
| 9 | Tablets | Ibuprofen | Acetaminophen | Pseudoephedrine |
| | | 400 mg | 325 mg | 30 mg |
| 10 | Tablets | Ibuprofen | Acetaminophen | Pseudoephedrine |
| | | 400 mg | 500 mg | 30 mg |
| 11 | Capsules | Ibuprofen | Acetaminophen | Pseudoephedrine |
| | | 400 mg | 325 mg | 30 mg |
| 12 | Capsules | Ibuprofen | Acetaminophen | Pseudoephedrine |
| | | 400 mg | 500 mg | 30 mg |
| 13 | Tablets | Ibuprofen | Acetaminophen | Pseudoephedrine |
| | | 600 mg | 325 mg | 30 mg |
| 14 | Capsules | Ibuprofen | Acetaminophen | Pseudoephedrine |
| | | 600 mg | 325 mg | 30 mg |
| 15 | Tablets | Ibuprofen | Acetaminophen | Pseudoephedrine |
| | | 200 mg | 325 mg | 60 mg |
| 16 | Tablets | Ibuprofen | Acetaminophen | Pseudoephedrine |
| | | 200 mg | 500 mg | 60 mg |
| 17 | Tablets | Ibuprofen | Acetaminophen | Pseudoephedrine |
| | | 400 mg | 325 mg | 60 mg |
| 18 | Tablets | Ibuprofen | Acetaminophen | Pseudoephedrine |
| | | 400 mg | 500 mg | 60 mg |
| 19 | Capsules | Ibuprofen | Acetaminophen | Pseudoephedrine |
| | | 200 mg | 325 mg | 60 mg |
| 20 | Capsules | Ibuprofen | Acetaminophen | Pseudoephedrine |
| | | 200 mg | 500 mg | 60 mg |
| 21 | Capsules | Ibuprofen | Acetaminophen | Pseudoephedrine |
| | | 400 mg | 325 mg | 60 mg |
| 22 | Capsules | Ibuprofen | Acetaminophen | Pseudoephedrine |
| | | 400 mg | 500 mg | 60 mg |
| 23 | Tablets | Ibuprofen | Acetaminophen | Pseudoephedrine |
| | | 200 mg | 325 mg | 120 mg |
| 24 | Tablets | Ibuprofen | Acetaminophen | Pseudoephedrine |
| | | 200 mg | 500 mg | 120 mg |
| 25 | Tablets | Ibuprofen | Acetaminophen | Pseudoephedrine |
| | | 400 mg | 325 mg | 120 mg |
| 26 | Tablets | Ibuprofen | Acetaminophen | Pseudoephedrine |
| | | 400 mg | 500 mg | 120 mg |
| 27 | Capsules | Ibuprofen | Acetaminophen | Pseudoephedrine |
| | | 200 mg | 325 mg | 120 mg |
| 28 | Capsules | Ibuprofen | Acetaminophen | Pseudoephedrine |
| | | 200 mg | 500 mg | 120 mg |
| 29 | Capsules | Ibuprofen | Acetaminophen | Pseudoephedrine |
| | | 400 mg | 325 mg | 120 mg |
| 30 | Capsules | Ibuprofen | Acetaminophen | Pseudoephedrine |
| | | 400 mg | 500 mg | 120 mg |
| 31 | Liquid | Ibuprofen | Acetaminophen | Pseudoephedrine |
| | | 100 mg/5 ml | 160 mg/5 ml | 7.5 mg/0.8 ml |
| 32 | Liquid | Ibuprofen | Acetaminophen | Pseudoephedrine |
| | | 40 mg/ml | 100 mg/ml | 7.5 mg/0.8 ml |
| 33 | Chewable | Ibuprofen | Acetaminophen | Phenylephrine |
| | tablets | 50 mg | 80 mg | 5 mg |
| 34 | Tablets | Ibuprofen | Acetaminophen | Phenylephrine |
| | | 400 mg | 325 mg | 10 mg |
| 35 | Tablets | Ibuprofen | Acetaminophen | Phenylephrine |
| | | 400 mg | 500 mg | 10 mg |
| 36 | Capsules | Ibuprofen | Acetaminophen | Phenylephrine |
| | | 400 mg | 325 mg | 10 mg |
| 37 | Capsules | Ibuprofen | Acetaminophen | Phenylephrine |
| | | 400 mg | 500 mg | 10 mg |
| 38 | Liquid | Ibuprofen | Acetaminophen | Phenylephrine |
| | | 100 mg/5 ml | 160 mg/5 ml | 10 mg |
| 39 | Liquid | Ibuprofen | Acetaminophen | Phenylephrine |
| | | 40 mg/ml | 100 mg/ml | 10 mg |
| 40 | Chewable | Ibuprofen | Acetaminophen | Pseudoephedrine |
| | tablets | 50 mg | 80 mg | 15 mg |
| | | | | Lysine |
| 41 | Chewable | Ibuprofen | Acetaminophen | Pseudoephedrine |
| | tablets | 100 mg | 120 mg | 15 mg |
| | | | | Lysine |
| 42 | Chewable | Ibuprofen | Acetaminophen | Pseudoephedrine |
| | tablets | 100 mg | 80 mg | 15 mg |
| | | | | Lysine |
| 43 | Chewable | Ibuprofen | Acetaminophen | Pseudoephedrine |
| | tablets | 100 mg | 160 mg | 15 mg |
| | | | | Lysine |
| 44 | Tablets | Ibuprofen | Acetaminophen | Pseudoephedrine |
| | | 200 mg | 325 mg | 30 mg |
| | | | | Lysine |
| 45 | Tablets | Ibuprofen | Acetaminophen | Pseudoephedrine |
| | | 200 mg | 500 mg | 30 mg |
| | | | | Lysine |
| 46 | Capsules | Ibuprofen | Acetaminophen | Pseudoephedrine |
| | | 200 mg | 325 mg | 30 mg |
| | | | | Lysine |
| 47 | Capsules | Ibuprofen | Acetaminophen | Pseudoephedrine |
| | | 200 mg | 500 mg | 30 mg |
| | | | | Lysine |
| 48 | Tablets | Ibuprofen | Acetaminophen | Pseudoephedrine |
| | | 400 mg | 325 mg | 30 mg |
| | | | | Lysine |
| 49 | Tablets | Ibuprofen | Acetaminophen | Pseudoephedrine |
| | | 400 mg | 500 mg | 30 mg |
| | | | | Lysine |
| 50 | Capsules | Ibuprofen | Acetaminophen | Pseudoephedrine |
| | | 400 mg | 325 mg | 30 mg |
| | | | | Lysine |
| 51 | Capsules | Ibuprofen | Acetaminophen | Pseudoephedrine |
| | | 400 mg | 500 mg | 30 mg |
| | | | | Lysine |
| 52 | Tablets | Ibuprofen | Acetaminophen | Pseudoephedrine |
| | | 600 mg | 325 mg | 30 mg |
| | | | | Lysine |
| 53 | Capsules | Ibuprofen | Acetaminophen | Pseudoephedrine |
| | | 600 mg | 325 mg | 30 mg |
| | | | | Lysine |
| 54 | Tablets | Ibuprofen | Acetaminophen | Pseudoephedrine |
| | | 200 mg | 325 mg | 60 mg |
| | | | | Lysine |
| 55 | Tablets | Ibuprofen | Acetaminophen | Pseudoephedrine |
| | | 200 mg | 500 mg | 60 mg |
| | | | | Lysine |
| 56 | Tablets | Ibuprofen | Acetaminophen | Pseudoephedrine |
| | | 400 mg | 325 mg | 60 mg |
| | | | | Lysine |
| 57 | Tablets | Ibuprofen | Acetaminophen | Pseudoephedrine |
| | | 400 mg | 500 mg | 60 mg |
| | | | | Lysine |
| 58 | Capsules | Ibuprofen | Acetaminophen | Pseudoephedrine |
| | | 200 mg | 325 mg | 60 mg |
| | | | | Lysine |
| 59 | Capsules | Ibuprofen | Acetaminophen | Pseudoephedrine |
| | | 200 mg | 500 mg | 60 mg |
| | | | | Lysine |
| 60 | Capsules | Ibuprofen | Acetaminophen | Pseudoephedrine |
| | | 400 mg | 325 mg | 60 mg |
| | | | | Lysine |
| 61 | Capsules | Ibuprofen | Acetaminophen | Pseudoephedrine |
| | | 400 mg | 500 mg | 60 mg |
| | | | | Lysine |
| 62 | Tablets | Ibuprofen | Acetaminophen | Pseudoephedrine |
| | | 200 mg | 325 mg | 120 mg |
| | | | | Lysine |
| 63 | Tablets | Ibuprofen | Acetaminophen | Pseudoephedrine |
| | | 200 mg | 500 mg | 120 mg |
| | | | | Lysine |
| 64 | Tablets | Ibuprofen | Acetaminophen | Pseudoephedrine |
| | | 400 mg | 325 mg | 120 mg |
| | | | | Lysine |
| 65 | Tablets | Ibuprofen | Acetaminophen | Pseudoephedrine |
| | | 400 mg | 500 mg | 120 mg |
| | | | | Lysine |
| 66 | Capsules | Ibuprofen | Acetaminophen | Pseudoephedrine |
| | | 200 mg | 325 mg | 120 mg |
| | | | | Lysine |
| 67 | Capsules | Ibuprofen | Acetaminophen | Pseudoephedrine |
| | | 200 mg | 500 mg | 120 mg |
| | | | | Lysine |
| 68 | Capsules | Ibuprofen | Acetaminophen | Pseudoephedrine |
| | | 400 mg | 325 mg | 120 mg |
| | | | | Lysine |
| 69 | Capsules | Ibuprofen | Acetaminophen | Pseudoephedrine |
| | | 400 mg | 500 mg | 120 mg |
| | | | | Lysine |
| 70 | Liquid | Ibuprofen | Acetaminophen | Pseudoephedrine |
| | | 100 mg/5 ml | 160 mg/5 ml | 7.5 mg/0.8 ml |
| | | | | Lysine |
| 71 | Liquid | Ibuprofen | Acetaminophen | Pseudoephedrine |
| | | 40 mg/ml | 100 mg/ml | 7.5 mg/0.8 ml |
| | | | | Lysine |

### Example 3

In a proof-of-concept, open study, a combination of the following was administered on 24 different times to a study subgroup of n=2:
1. 1 Acetaminophen 500 mg tablet,
2. 1 Ibuprofen 200 mg tablet, and
3. 1 Pseudoephedrine HCL 30 mg tablet (Sudafed®)

This combination of drugs was ingested for the treatment of various symptoms including but not limited to: headache, nasal congestion and sinus pressure. The administration of this combination of drugs demonstrated safety, tolerability and efficacy.

### Example 4

In a proof-of-concept, open study, a combination of the following was administered on 24 different times to a study subgroup of n=2:
1. 1 Acetaminophen 500 mg tablet,
2. 1 Ibuprofen 200 mg tablet, and
3. 1 Phenylephrine HCL 10 mg tablet (Sudafed PE®)

This combination of drugs was ingested for the treatment of various symptoms including but not limited to: headache, nasal congestion and sinus pressure. The administration of this combination of drugs demonstrated safety, tolerability and efficacy.

1. A pharmaceutical formulation comprising (a) a phenylpropionate, (b) an acetanilide, and (c) a sympathomimetic drug or a prodrug form of a sympathomimetic drug; and a pharmaceutically acceptable carrier.

2. (canceled)

3. A pharmaceutical formulation of claim 1, wherein said phenylpropionate is ibuprofen.

4. (canceled)

5. A pharmaceutical formulation of claim 1, wherein said acetanilide is acetaminophen.

6. A pharmaceutical formulation of claim 1, wherein said sympathomimetic drug or prodrug form of a sympathomimetic drug is pseudoephedrine, or a prodrug form of pseudoephedrine.

7.-10. (canceled)

11. A pharmaceutical formulation of claim 1, wherein said sympathomimetic drug is phenylephrine.

12. The use of a pharmaceutical formulation of claim 1 for the treatment of at least one of pain, headache, fever, nasal congestion, sinus congestion, runny nose, sore throat, myalgia, or otic barotrauma.

13. A dosing regimen comprising (a) a phenylpropionate, (b) an acetanilide, and (c) a sympathomimetic drug or a prodrug version of a sympathomimetic drug.

14. (canceled)

15. A dosing regimen of claim 13, wherein said phenylpropionate is ibuprofen.

16. (canceled)

17. A dosing regimen of claim 13, wherein said acetanilide is acetaminophen.

18. A dosing regimen of claim 13, wherein said sympathomimetic drug is pseudoephedrine, or a prodrug version of pseudoephedrine.

19-22. (canceled)

23. A dosing regimen of claim 13, wherein said sympathomimetic drug is phenylephrine.

24. A method of treating one or more of symptoms from the group consisting of pain, headache, fever, nasal congestion, sinus congestion, runny nose, or otic barotrauma, said method comprising administering to a mammalian subject suffering from said condition a formulation of claim 1.

25. A method of treating one or more symptoms from a group consisting of pain, headache, fever, nasal congestion, sinus congestion, runny nose, sore throat, myalgia, or otic barotrauma, said method comprising administering to a mammalian subject suffering from said condition a dosing regimen of claim 13.

26. A kit comprising three or more pharmaceutical formulations, wherein the first pharmaceutical formulation comprises a phenylpropionate and a pharmaceutically acceptable carrier, the second pharmaceutical formulation comprises an acetanilide and a pharmaceutically acceptable carrier, and the third pharmaceutical formulation comprises a sympathomimetic drug or a prodruq version of a sympathomimetic drug and a pharmaceutically acceptable carrier.

27-35. (canceled)

36. A kit of claim 26, wherein said phenylpropionate is ibuprofen, said sympathomimetic drug is pseudoephedrine, a prodrug version of pseudoephedrine, or phenylephrine, and said acetanilide is acetaminophen, further comprising instructions for administering said combination for the treatment of at least one of pain, headache, fever, nasal congestion, sinus congestion, runny nose, sore throat, myalgia, or otic barotrauma.

37. A pharmaceutical formulation comprising a phenylpropionate and a sympathomimetic drug or a prodrug version of a sympathomimetic drug and a pharmaceutically acceptable carrier.

38. A pharmaceutical formulation of claim 37, wherein said phenylpropionate is ibuprofen.

39. A pharmaceutical formulation of claim 37, wherein said sympathomimetic drug is pseudoephedrine or a prodrug version of pseudoephedrine.

40-42. (canceled)

43. A pharmaceutical formulation comprising an acetanilide and a sympathomimetic drug or a prodrug version of a sympathomimetic drug and a pharmaceutically acceptable carrier.

44. A pharmaceutical formulation of claim 43, wherein said acetanilide is acetaminophen.

45. A pharmaceutical formulation of claim 43, wherein said sympathomimetic drug is pseudoephedrine or a prodrug version of pseudoephedrine.

46-48. (canceled)
